(19) United States
(12) Patent Application Publication
(10) Pub. No.: US 2007/0191769 A1
(43) Pub. Date: Aug. 16, 2007

(54) Needle uncap/recap safety device

(76) Inventor: Eric John Meittunen, Hibbing, MN (US)

Correspondence Address: Warren A. Reiter, 464 N. David Street, Nevada, MO, 64772, US

(21) Appl. No.: 11/351,542

(22) Filed: Feb. 10, 2006

(51) Int. Cl. A61M 5/00

(52) U.S. Cl. 604/110

(57) ABSTRACT

A device and a method that allows a user to safely uncap or recap the protective cap, cover, or shield of a sharps instrument with one-hand and without injury in multiple settings. The device includes a removable protective shield that attaches to a main housing with a cone shaped threaded capture-release mechanism, a handle, and an adjustable multi-surface attachment means to protect the user from sharp or needle-stick while capping or recapping sharp instruments. The removable protective shield has an orifice that effectuates the insertion of the needle through the shield to the reach main housing capture-release mechanism while protecting the users hand. The present invention also includes a method by which the device is to be used for the safe removal and replacement of protective, cap, cover, or sheath of a sharp instrument as the main housing contains a threaded capture-release mechanism that grips the protective cap, cover, or sheath when twisted. The protective cap, cover, or shield is released when twisted in the opposite direction.

### FEDERALLY SPONSORED RESEARCH

Not Applicable

### SEQUENCE LISTING OR PROGRAM

Not Applicable

### BACKGROUND

1. Field of Invention

This invention is a recapping and uncapping safety device that is used to remove and replace the protective cap, cover, or sheath of a needle or other sharp instrument typically found in healthcare, laboratories, or veterinary facilities.

Healthcare staffs use thousands of syringes on a daily basis as a part of their patient care activities. Hypodermic needles are used to give injections and draw blood from patients, however these needles can become contaminated with any number of infectious and potentially lethal diseases. The threat of accidental puncture wounds from contaminated needles, scalpels, and other sharp instruments poses a significant safety risk to medical personnel, laboratory technicians, hospital cleaning staff, waste disposal workers and the general public.

The uncap/recap safety device primarily consists of a sharps injury prevention shield to protect items located behind the device, a main housing apparatus in which to place the needle or other sharp instrument, and a capture-release mechanism to fit various size syringe or sharp instrument protective caps, sheaths, or covers. The uncap/recap safety device has an adjustable multi-surface attachment means that is attached to the main housing with pins or other means to allow for device adjustment. The main housing is the appropriate size and shape to provide for a handle that assists the user to place the device when preparing to use the device. The capture release mechanism allows the user to place inside the main housing a needle or sharp instrument with a protective cover and with a twisting motion hold taught the protective cover whereby the needle or sharp instrument may be pulled from the protective cover, used in the healthcare process, safely reinserted into the needle or sharp instrument protective cover, and then freed from the device via a twisting motion in the opposite direction. The needle or sharp protective cover uncap/recap method may be accomplished safety with one hand and in a convenient location in order to reduce the likelihood of an injury.

2. Description of Prior Art

Approximately 600 to 800 thousand needle-stick or sharps injuries occur each year to healthcare employees in the United States, additionally, these injuries have been documented in the transmission of life threatening blood-borne pathogens like HIV and Hepatitis. Workers may also suffer from adverse side effects of drugs used for post-exposure prophylaxis and from psychological stress due to the threat of infection following an exposure incident.

Even though the law mandates the use of safety devices to reduce the potential for exposures related to needle-stick and other sharp instruments in healthcare organizations, data indicates that the number of needle-stick injuries has not significantly declined. Compounding the scenario is that the number of healthcare employees in the United States is predicted to grow beyond the present 12 million staff. The future result is a potential for an increase in exposures, the associated healthcare occupational injuries, and the potential for the transmission of infection. Thus more work needs to be accomplished in order to develop devices that provide safety for healthcare staff and provide for a fit within the healthcare systems.

Numerous devices have emerged which to help decrease the chance of a needle-stick or sharp instrument injury for healthcare staff whilst accomplishing a variety of tasks including giving injections and blood draws. The present art associated with syringe needle safety includes a type of equipment add-ons or variants of syringes similar to those described in U.S. Pat. No. 6,884,224 to Dalton (2005); Needle capping devices like those described in U.S. Pat. No. 425,197 to Corner (2000); Recapping devices that are limited to needle hub systems comparable to U.S. Pat. No. 5,356,385 to Latini (1994); Needle capping and for transport of the syringe or sharp instrument equivalent to those noted in U.S. Pat. No. 5,348,543 to Talley (1994), and more complex mechanical means for capping and uncapping syringe needles analogous to those depicted in U.S. Pat. No. 5,209,738 to Bruno (1993).

Patents to Dalton, U.S. Pat. No. 6,884,224 and Smith, U.S. Pat. No. 334,979 are various types of add-on devices or new syringe designs that must be attached to the uncapped needle of a syringe with two hands in order to be operated. These devices have several limitations such as placing the user of the devices at risk of injury by having to prepare the device with an unsafe exposed needle and only for single use thus increasing the expense for healthcare providers, in addition to the fact that a healthcare organization may have to purchase new syringes at an added expense. These types of devices also appear to be complicated to use, as successful use is dependant upon factors such as adhesive materials, springs, and caps.

Patents to Corner, U.S. Pat. No. 425,197; Tucker, U.S. Pat. No. 370,062; and Drummond, U.S. Pat. No. 355,182 include capping devices ornamental designs consisting of a pyramid and tube like structures that show an object for which a needle with cap may be stood-up vertically from the device that perhaps the needle with cap could be inserted. These designs are limited in scope for gripping, release, or removal of the needle cap and the surface adherence in order to attach the device for use. Additional limitations entail the limits of syringe size capabilities and the appropriate workstation positioning for the devices to optimize human factor issues during use. These capping devices also only provide for a means to hold the syringe needle cap in preparation for insertion of the needle into the cap and do not include the process needs for uncapping in their design as the user must still risk injury while using two hands to uncap the syringe needle. The current invention is designed to allow for one hand needle or sharp instrument uncapping and recapping, additionally is simple to use.

Other patents depict methods to recap syringes restricted to needle hub systems that facilitate the removal of needle hub assemblies from syringe bodies after use by placing the needle syringe hub in the device opening as those described in patents to Schneider, U.S. Pat. No. 504,511; Latini, U.S. Pat. No. 5,356,385; and Daly, U.S. Pat. No. 5,356,383. These devices have many complicated parts including actuators, electric motors, switches, transformers, printed circuit boards, and a removal heads, springs, rotor plates, gripper blades, torque springs, and a release arms. Typically once a syringe with hub assembly is placed in the opening of the device, the user then pushes an actuator button on the exterior of the housing in order to start the machine and turn the syringe hub assembly that is then removed from the syringe. The needle hub removal devices are limited in their scope via heavy weight, cost to produce, are cumbersome to operate, and are restricted to be used only for syringes with hub assemblies. The present invention differs in that the design is for the recapping or uncapping of any syringe protective cover and not just the removal of those hub assemblies, in addition to a more straightforward design.

Further patents illustrate methods and devices to cap syringes for transport such as patents to Talley, U.S. Pat. No. 5,348,543; Eckels, U.S. Pat. No. 5,347,078; and Pedicano, U.S. Pat. No. 4,623,336. These devices incorporate tubular sleeves with an open end or a closed end in which to hold the needle cap with or without a syringe. The devices attempt to facilitate the safety for staff while transporting a syringe with the needle cap already in place on the syringe to a location where the syringe is to be used or disposed. The devices may also act as a holder for the needle cap once removed from a syringe using a two handed technique. The designs may offer vertical ribbing means to align with the syringe needle hub shape and block the rotation of the syringe needle hub during removal, have a door closure function to seal the carrier for disposal or offer a stretched rubber band over the top of the syringe to aid in the safe transport of the syringe. Unlike the design of the current invention these designs are limited in use for syringes with removable locking hubs and increase the injury risk during use as require two hands to operate or safely remove the needle from the syringe hub. Additional limitations for these devices include the restriction to solely the capping process and do not function for the uncapping process unlike the capabilities in the present invention. The present invention also has a threaded needle or sharp instrument capture-release mechanism in order to provide for a positive friction fit for the needle or sharp instrument protective cap or cover when compared to the vertical ribbing that solely prevent turning while in the device for the devices aforementioned.

Other patents describe manners and designs related to a mechanical means for capping and uncapping syringe needles for example patents to Steiner, U.S. Pat. No. 4,955,865 and Bruno, U.S. Pat. No. 5,209,738. These patents are operated to uncap and recap syringe needles with two hands by pushing the syringe needle cap into the device whilst simultaneously holding the device in the other hand allowing for the needle cap or needle to be inserted. Devices described in these patents may be operated with one hand if devices are mounted or two hands if not mounted. However these devices entail many parts that may inhibit long term function without device failures including spring members, triggers or actuators, ballast weight, folded foam strips, clips, and golf tee like stand mechanisms. Additional limitations of these designs include the use of complicated mechanisms and user adjustments to fit the various size syringes. These complicated user adjustments are associated with the devices need to be tailored to meet the functional aspects of various syringe needles including the forces required to uncap syringe needles, the lengths of the syringe needle caps, and the width of the syringe needle caps and may add to the misuse and a reduction in safety if maladjusted for particular applications. The present invention is designed to be more reliable, simple, and a true one-handed technique for needle or sharp instrument uncapping and recapping of the protective cover.

### SUMMARY OF THE INVENTION

The present invention is designed to provide a compromise between the needle reinsertion device, Corner Patent; the hypodermic needle guard, Talley; the hypodermic needle sheath holder, Latini; the needle unsheathing, resheathing, and handling apparatus, Steiner; and the apparatus for enabling safe recapping of hypodermic needles, Bruno. This invention is easy to produce, simple to use by medical personnel, not time consuming to prepare, and still provides protection for medical personnel from inadvertently injuring themselves with syringes or other sharp instruments during the protective cover uncapping and recapping process. Additionally the device provides for a cost effective, shielded, one-handed, uncapping and recapping process for multiple size and style syringe needle caps or covers in order to protect the user from a needle-stick injury. The design of the invention also enables healthcare personnel to more safely use

Other patents describe manners and designs related to a mechanical means for capping and uncapping syringe needles for example patents to Steiner, U.S. Pat. No. 4,955,865 and Bruno, U.S. Pat. No. 5,209,738. These patents are operated to uncap and recap syringe needles with two hands by pushing the syringe needle cap into the device whilst simultaneously holding the device in the other hand allowing for the needle cap or needle to be inserted. Devices described in these patents may be operated with one hand if devices are mounted or two hands if not mounted. However these devices entail many parts that may inhibit long term function without device failures including spring members, triggers or actuators, ballast weight, folded foam strips, clips, and golf tee like stand mechanisms. Additional limitations of these designs include the use of complicated mechanisms and user adjustments to fit the various size syringes. These complicated user adjustments are associated with the devices need to be tailored to meet the functional aspects of various syringe needles including the forces required to uncap syringe needles, the lengths of the syringe needle caps, and the width of the syringe needle caps and may add to the misuse and a reduction in safety if maladjusted for particular applications. The present invention is designed to be more reliable, simple, and a true one-handed technique for needle or sharp instrument uncapping and recapping of the protective cover.

### SUMMARY OF THE INVENTION

The present invention is designed to provide a compromise between the needle reinsertion device, Corner Patent; the hypodermic needle guard, Talley; the hypodermic needle sheath holder, Latini; the needle unsheathing, resheathing, and handling apparatus, Steiner; and the apparatus for enabling safe recapping of hypodermic needles, Bruno. This invention is easy to produce, simple to use by medical personnel, not time consuming to prepare, and still provides protection for medical personnel from inadvertently injuring themselves with syringes or other sharp instruments during the protective cover uncapping and recapping process. Additionally the device provides for a cost effective, shielded, one-handed, uncapping and recapping process for multiple size and style syringe needle caps or covers in order to protect the user from a needle-stick injury. The design of the invention also enables healthcare personnel to more safely use the existing needle or sharp instrument equipment that is presently within the typical healthcare organization. The safety of the medical personnel is the primary means for the invention and offers a marked reduction in the potential for sharps or needle related injury when accomplishing a needle or sharp instrument protective cap or cover uncapping or recapping process with the use of only one hand without the need for a triggering or actuator means as compared to the Steiner patent. The present invention includes consideration for the human factors issues of the medical personnel with a positive friction fit capture-release mechanism that is designed to fit the various sizes of syringe and sharp instruments used in healthcare organizations as compared to the Bruno Patent that requires intricate adjustments for different size syringe caps. The invention also provides for a means to both safely uncap and recap a hypodermic needle with one hand operation as compared to the Corner and Talley Patents that are limited in use for only needle capping processes. The design of the present invention promotes simplicity and ease of use for the medical personnel by utilizing significantly less complicated parts like a plurality of gripping blades and actuating mechanisms than the Latini Patent. The present invention design also incorporates a trouble-free twisting motion in one direction to grip the syringe needle or sharps instrument protective cover or cap and a twisting motion in the opposite direction order to loosen or free the protective cap or cover. The user-friendly twisting motion for the capture-release apparatus to grip the needle or sharp instrument protective cap or cover is significantly easier to operate in the present invention than the aforementioned patent designs.

More particularly the device consists of a sharps protection shield to prevent items or hands located behind the device from being poked. The sharps protection shield is the appropriate size and shape to cover the medical personnel's hand in order to prevent injuries if the device is held during use. The sharps injury prevention shield may be detached during one-handed use as the device is mounted to a workstation surface depending upon safety and user preference, and the human factor issues involved in the application. An additional feature of the shield is that it provides for a focal point on the center syringe needle/cap entry point during use, thus facilitating the proper alignment of the syringe with or without needle cap in the center entry portal for the capture-release mechanism. The shield has a raised outer-edge about the parameter when placed facing the user in order to prevent the needle from slipping off the shield. The shield also offers barrier protection to prevent items located behind the device from being poked with the needle. The main surface of the shield is generally smooth and may have an ornamental design, shape, or text applied as needed to enhance safety, performance, or usability.

The device has a main housing apparatus that is large enough to allow for a built-in handle. This handle facilitates placement, carry, and transportation. The main housing contains the capture-release apparatus for the needle syringe or sharp instrument protective cap or cover to be threaded and gripped in the device via a friction fit means. The design length, width, and shape of the main housing are calculated to offer adequate size fit for the variety of syringe or sharp instrument protective caps or covers available. The main housing length design also considers the space to reach the capture-release mechanism with various styles of syringes while still able to use the protective shield for use of syringes that have short syringe needle caps. In addition the main housing bottom surface has a tiered stance adjustment feature that allows the user to properly angle or position the face of the device in a most convenient manner for use while at either a sitting or standing workstation.

This apparatus is designed to fit for multiple size syringe or sharp instrument protective caps, sheaths, or covers. The interior of the capture-release apparatus is cone-shaped with has ridges located around the interior cone parameter, similar to a threaded mechanism like the inside of a nut-screw operation. This cone-shape when combined with the aspects of the interior parameter ridge threads offer adequate friction to hold the cap of a syringe or protective cover of a sharp instrument when the user twists the syringe needle cap, sheath, or cover during insertion into the mechanism. Additionally the apparatus is designed to release the syringe or sharp instrument protective cap, sheath, or cover when twisted in the opposite direction. This feature eliminates the need to use two hands during the syringe uncapping or recapping process, thus promoting the safety of the healthcare staff during use as to not no have to hold, trigger, or fuss with complicated mechanical means is needed to hold/release the syringe needle cap, sheath, or cover.

The method to operate the capture-release mechanism involves aligning the needle cap of the syringe or sharp instrument to enter the portal of the capture-release mechanism. Slide the syringe needle protective cover or cap into the capture-release mechanism until friction is apparent. Hold the syringe or sharp instrument protective cover or cap as needed depending on the style of the syringe. Rotate the syringe or sharp instrument and/or protective cover or cap until friction stops the rotation. Grip the syringe or sharp instrument barrel away from the needle sharp and withdraw the syringe needle from the device. Healthcare personnel may then use the syringe as needed. To recap the syringe sharp instrument protective cover or cap align the syringe needle hold the syringe with one hand and point the needle or sharps end toward the shield of the device. Align the needle or sharp end of the instrument to enter the portal of the capture-release mechanism that contains the syringe cap. Slide the syringe needle or sharp instrument into the protective cover or cap being held by the device until fully fit. Hold the syringe or sharp instrument and/or protective cap as needed depending on the style of the syringe. Rotate the syringe or sharp instrument and/or protective cover or cap in the opposite direction as used to remove the protective cover or cap until friction is felt free and the syringe and cap may be removed.

The design of the present invention includes an adjustable multi-surface attachment means. The main housing is connected to the multi-surface attachment means via, but not limited to, a pin that extends through both the orifice located on the bottom of the main housing and the top of the multi-surface attachment means. The multi-surface attachment means entails, but is not limited to, a sloped round outer shell with an inner membrane of a different material than the embodiment. The multi-surface attachment mechanism supports the features of a suction cup serving to easily attach or detach the device to a variety of surfaces. Attachment of the present invention may also consist of Velcro, glue, or screws to the workstation surface.

The device may be made from but not limited to multiple plastic properties, metal, or ceramic with pins.

### BRIEF DESCRIPTION OF THE DRAWINGS

Drawing Figures

FIG. 1 illustrates a side view of the preferred design of the invention.

FIG. 2 illustrates a top view of the preferred design of the invention.

### DETAILED DESCRIPTION OF THE PREFERRED EMBODIMENT

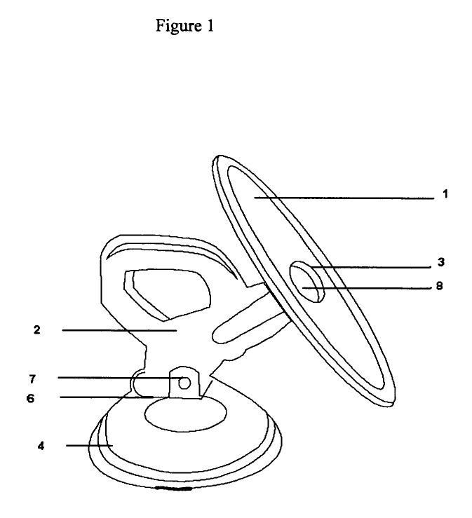

FIG. 1 shows the major advantage of this invention to be a main housing apparatus 2 that provides the enclosure for the syringe or sharp instrument protective cap or cover capture-release mechanism 8. The main housing apparatus is between 1 to 12 centimeters in length and 0.4 millimeters to 5.5 centimeters in diameter. The main housing apparatus 2 provides a means to attach the sharps protective shield 1 to the device. The shield 1 also has a raised outer-edge 5 about the parameter when placed facing the user in order to prevent the needle from slipping off the shield. The shield also offers barrier to prevent items located behind the device from being poked with the needle. The main surface of the shield 1 is generally smooth and may have an ornamental design, shape, or text applied as needed to enhance safety, performance, or usability. The shield 1 is between 3 to 17 centimeters in diameter and between 0.3 mm to 5.3 centimeters in thinness with an orifice that is between 0.3 millimeters to 5 centimeters. Additionally the main housing is connected to the multi-surface attachment means 4 in order to be positioned on a workstation. The main housing 2 is cylindrical in shape and also provides for a handle that assist the user to place the device when using the multi-surface attachment means 4. The design length, width, and shape of the main housing 2 is calculated to offer adequate size fit for the variety of syringe caps available. The main housing length design also considers the space to reach the interior of the capture-release mechanism 8 with various styles of syringes while still be able to use the protective shield 1 for use of syringes that have short syringe needle caps. The capture-release mechanism 8 is between 2 millimeters to 15.7 centimeters in length with threads ranging from 0.2 millimeter to 3.1 centimeters in width. The distance from the opening of the capture release mechanism to the threads may range from 0 millimeters to 11 centimeters. In addition the base of the main housing has a tiered stance adjustment feature 6 that allows the user to properly angle or position the face of the device in a most convenient manner for use while at either a sitting or standing workstation. The angle of the tiered stance adjustment feature to the mounting surface 6 may range from 0 to 180 degrees. The main housing 2 is connected to the multi-surface attachment means 4 via, but not limited a pin, 7 that extends through both the orifice located on the bottom of the main housing and the top of the multi-surface attachment means. The multi-surface attachment means 4 entails a sloped round outer shell with an inner membrane of a different material than the embodiment. The multi-surface attachment mechanism 4 supports the features of a suction cup serving to easily attach or detach the device to a variety of surfaces. The attachment may also consist of Velcro, glue, or screws to the workstation surface.

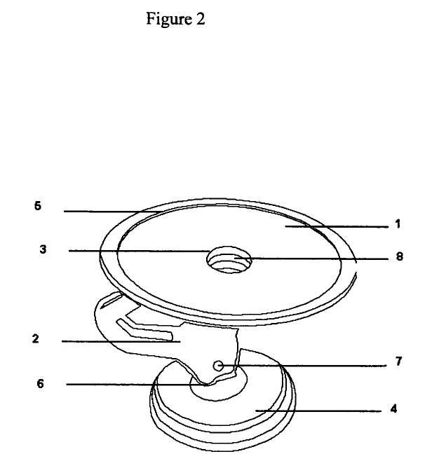

FIG. 2 shows the interior of the main housing 2 that contains the capture-release mechanism 8. This interior of the capture-release mechanism 8 is accessed via the circular or other preferred shape opening in the face of the shield 3. The capture-release mechanism 8 located within the main housing 2 may also be used without the protective shield 1 depending on safety, user preferences, or workstation design. The interior of the capture-release mechanism 8 is an apparatus that has cone-shaped ridges located around the interior cone parameter, similar to a threaded mechanism like the inside of a nut-screw operation. This cone-shape when combined with the aspects of the interior parameter ridge threads offer adequate friction to hold the cap of a syringe when the user twists the syringe needle cap, sheath, or cover during insertion into the mechanism and the ability to release the syringe needle cap, sheath, or cover when twisted in the opposite direction. This feature eliminates the need to use two hands during the syringe uncapping or recapping process, thus promoting the safety of the healthcare staff during use as not holding, triggering, or complicated mechanical means is needed to hold/release the syringe needle cap, sheath, or cover.

What is claimed is:

1. An apparatus to selectively retain a syringe needle or sharp instrument protective cap, cover, or sheath for safely uncapping and recapping a syringe needle or sharp instrument device with the use of one hand comprising;
a main housing that has a top and a bottom end and at least one opening at the top of said main housing;
said main housing further has incorporated within a handle or loop for user positioning, carry, transfer;
incorporated within the main housing opening with a capture-release mechanism for multiple size syringe needle or sharp instrument protective caps, covers, or sheaths;
attached to the top of the main housing is a fixed or removable sharps protection shield with an opening for access to the main housing capture release mechanism that protects the users hand from sharp point;
and pivotally attached to the bottom is a mounting device.

2. The apparatus of claim 1 where the capture release mechanism is cone shaped.

3. The apparatus of claim 1 where the main housing is between 1 to 12 centimeters in length and 0.4 millimeters to 5.5 centimeters in diameter.

4. The apparatus of claim 1 where the capture release mechanism has either a plural or singular thread design with threads ranging from 0.2 millimeters to 3.1 centimeters in width and said threads are position within 0 to 11 centimeters from main housing opening in order to accommodate a variety of size and shapes of syringes or medical sharp device protective covers.

5. The apparatus of claim 1, where the mounting device is attached to main housing in order to allow the angle of the tiered stance to be varied from 0 to 180 degrees;
adjustable in height, size, width, and expansion in order to accommodate the workstation;
said mounting device may mounted by a suction cup design or more permanently by glue or screws.

6. The apparatus of claim 1 where the fixed or removable sharps protective shield;
said shield having a raised outer-edge about the circumstance of side of the shield facing the user in order to prevent the needle from slipping off the shield;
said shield main surface is generally smooth and may have an ornamental design, shape, or text applied as needed to enhance safety, performance, or usability;
said shield is between 3 to 17 centimeters in diameter; between 0.3 mm to 5.3 centimeters in thickness; including at least one orifice that is between 0.3 millimeters to 5 centimeters in diameter to allow.

7. An apparatus to selectively retain a syringe needle or sharp instrument protective cap, cover, or sheath for safely uncapping and recapping a syringe needle or sharp instrument device with the use of one hand comprising;
a cone capture release mechanism having either a plural or singular thread design with threads ranging from 0.2 millimeters to 3.1 centimeters in width and said threads are position within 0 to 11 centimeters from an opening in order to accommodate a variety of size and shapes of syringes or medical sharp device;
said capture release mechanism is incorporated into workstation design either fixed or portable like sharps containers, desks, beds, laboratory ventilation hood or similar device;
with a fixed or removable sharps protection shield with an opening for access to the capture release mechanism to protect the users hand from sharp point;
pivotally attached to the bottom is a mounting device;
and may or may not include a handle.

8. A method for safely capping and recapping protective caps, covers, sheaths for syringe and sharp instruments consisting of;
the user inserting the syringe or sharp instrument with protective cap into a device capture release mechanism;
twists the syringe or sharp instrument in a clockwise direction to secure the protective cap, cover, or sheath within said capture release mechanism;
the user removes the syringe or sharp instrument therefore capturing the protective cap, cover, or sheath in said capture release mechanism;
the user reinserts the syringe or sharp instruments back into the protective cap, cover, or sheath;
the user twists the syringe or sharp instrument in a counterclockwise direction to release the syringe or sharp instrument and protective cap, cover, or sheath from said capture release mechanism;

9. The method described in claim 8 where the protective cap, cover, or sheath is captured in the capture release mechanism by twisting the syringe or sharp instrument in a counterclockwise direction and in a clockwise direction to release protective cap, cover, or sheath from said capture release mechanism.
